(19) United States
(12) Patent Application Publication
(10) Pub. No.: US 2002/0102548 A1
(43) Pub. Date: Aug. 1, 2002

(54) Methods for the preparation and use of internal standards for nucleic acid amplification assays

(75) Inventors: Klaus Zimmermann, Vienna (AT); Peter Turecek, Klosterneuburg (AT); Hans-Peter Schwarz, Vienna (AT); Manfred Rieger, Ganserndorf (AT)

Correspondence Address: OPPENHEIMER WOLFF & DONNELLY LLP, 840 NEWPORT CENTER DRIVE, SUITE 700, NEWPORT BEACH, CA, 92660, US

(73) Assignee: Baxter Aktiengesellschaft

(21) Appl. No.: 09/746,547

(22) Filed: Dec. 20, 2000

(30) Foreign Application Priority Data
Dec. 22, 1999 (AT) A2170/99

(51) Int. Cl. C12Q 1/68; C07H 21/04; C12P 19/34

(52) U.S. Cl. 435/6; 435/91.2; 536/24.3

(57) ABSTRACT

Internal nucleic acid standards for nucleic acid amplification assays are provided. Specifically, internal nucleic acid standards are provided that are prepared using non-recombinant DNA technology. These internal nucleic acid standards are generally chemically synthesized and have a minimum size of approximately 90 nucleic acid bases. Also provided are internal nucleic acid standards prepared using non-recombinant DNA techniques that are single stranded nucleic acids.

### CROSS REFERENCE TO RELATED APPLICATIONS

This application claims the benefit of Austrian patent application No. A2170/99, filed Dec. 22, 1999, which is herein incorporated by reference in its entirety.

### FIELD OF THE INVENTION

The present invention relates to a process for the detection and quantification of nucleic acids in a sample by using internal nucleic acid standards. Specifically, the present invention provides internal controls for nucleic acid amplification assays that are derived by chemical synthesis not through recombinant DNA technology techniques. More specifically, the present invention relates to chemically synthesized single stranded nucleic acid constructs useful as internal controls for nucleic acid amplification assays.

### BACKGROUND OF THE INVENTION

The routine determination of nucleic acids in biological samples is of ever-increasing importance both in research and in the applied pharmaceutical and medical industry. It is of particular importance that biological samples intended for in vivo use be screened for nucleic acids associated with transmissible infectious agents. Many of these infectious agents are either uncultivable, or extremely difficult to culture. Moreover, immunoassay based detection techniques often lack the sensitivity require to detect transmissible infectious agents at extremely low concentrations. Therefore, nucleic acid amplification assays have become the detection method of choice.

Recently, the number of biotechnology derived therapeutics have increased significantly. Biotechnology derived therapeutics include, but are not limited to recombinant growth factors, recombinant blood coagulation factors and recombinant vaccines. These recombinant materials are produced in in vitro cell culture bioreactors using recombinant DNA technology. The World health Organization (WHO) and United States Food and Drug Administration (US FDA) have expressed concern that residual heterologous DNA (a DNA construct containing DNA from two or more different species) may pose unknown risks. Therefore, these agencies have set limits on the amount of heterologous DNA that can be present in a biotechnology derived therapeutic. The WHO requires that the quantity of heterologous contaminating DNA has to be below 100 pg per dose, whereas the US FDA permits a maximum of 10 pg of DNA per dose. Consequently, all biotechnology-derived products must be tested for residual heterologous DNA content using nucleic acid detection techniques.

Moreover, in addition to the need to screen biological specimens and biotechnology derived therapeutics using DNA detection techniques, the burgeoning area of genomic and genetic typing has even further increased the demand for rapid, sensitive and specific nucleic acid detection assays.

Classical methods for nucleic acid detection such as membrane capture hybridization assays (dot blots) have a maximum sensitivity of approximately 5 pg of DNA. However, these assays are tedious to perform and are notoriously difficult to accurately quantify. Moreover, dot blots are generally manual methods that are not suited for large-scale screening. Consequently, the polymerase chain reaction (PCR) assay as described generally in United States patent application numbers U.S. Pat. Nos. 4,683,195 and 4,683,202 is the preferred technique. Since the development of the original PCR technique in the 1980's, many new modification and improvements have been developed. These include, but are not limited to, the reverse transcriptase PCR (RT-PCR), the ligase PCR (LCR) and the Taqman® PCR (referred to hereinafter collectively as either PCR or nucleic acid amplification assays).

Nucleic acid amplification assays possess exceptional sensitivity and specificity. However, all nucleic acid amplification assays are limited in that samples derived from biological fluids such as blood or cell lysates often contain impurities that inhibit the amplification reaction and lead to false negative results. In addition, false negative results arise as a result of nucleic acid loss in the extraction process. Moreover, technical errors during sample preparation and nucleic acid extraction may also contribute to false negative results. Therefore, internal amplification controls may be added prior to the amplification reaction, or prior to the extraction or purification of the nucleic acid, to permit the recognition of false negative results prior to reporting.

Internal controls for PCR assays generally consisting of nucleic acid molecules that contain a detectable nucleic acid sequence (standard oligonucleotide) that is different form the nucleic acid the assay is designed to detect (target oligonucleotide). The standard oligonucleotide is generally flanked by the same primer sequence used to initiate amplification of the target oligonucleotide. When the PCR assay is performed correctly, the standard oligonucleotide will be detected during post amplification analysis. Samples having standard oligonucleotide and target oligonucleotide detected are true positives, samples having only standard oligonucleotide detected are true negatives, samples having only target oligonucleotide detected are false positives and samples having no detectable amplified standard oligonucleotide are false negatives.

Generally, internal standards are made using recombinant DNA techniques that result in a recombinant DNA molecules having the standard oligonucleotide sequence flanked by primers consisting of duplex-DNA. However, standard oligonucleotide preparation using recombinant DNA techniques is a costly, time-consuming process and requires skilled scientists. Tedious internal standard preparation procedures currently being used represent a considerable factor in terms of assay time and cost.

Therefore, there is a need for developing internal standards for nucleic acid amplification assays that can be performed quickly and inexpensively without sacrificing assay specificity or sensitivity.

### BRIEF SUMMARY OF THE INVENTION

The present invention provides novel methods for the preparation of nucleic acid amplification assays that address the aforementioned and other problems associated with presently available techniques. In one embodiment of the present invention a process for the detection and quantification of nucleic acids in a sample is provided that consists of adding a chemically synthesized oligonucleotide with a size of at least 90 bases (pairs) to a sample as an internal standard. The sample is then amplified using methods known to those skilled in the art including, but not limited to PCR.

The desired sequence of the internal standard in accordance with the invention is hereby made available in a simple manner by means of chemical synthesis. Numerous firms already exist that offer the synthesis of such oligonucleotides within an extremely short time and at very efficient costs. Oligonucleotide synthesizers such the the Synthesizer 2000 MWG-Biotech AG, Ebenberg (Germany) are also readily available. The quantification of the oligonucleotides synthesized usually takes place automatically in the synthesizer or it is already provided by the synthesizer system. In addition, this quantification is not interfered by the presence of proteins which can be eliminated only as a result of time-consuming, expensive purification and extraction processes in the case of the recombinant DNA techniques n the prior art.

Surprisingly, the process in accordance with the invention is superbly well suited to large-scale industrial use and to comprehensive medical screening tests as a result of the provision of chemically synthesized oligonucleotides with a length of at least 90 bases pairs (bp) as standards, and it specifically offers advantages relative to biologically prepared samples in the organizational and preparative area.

Neither the occurrence of problems with nonspecific bands nor impediments as a result of the formation of primer-dimers have been found in the case of using chemically synthesized oligonucleotide standards starting from a length of approximately 90 bp. In accordance with the invention, use is preferably made, in particular, of standards of a length of 100 to 400 bp and especially 105 to 200 bp.

The term nucleic acid amplification is to be understood primarily to signify processes that are based on the technology that has been developed by Mullis et al. U.S. Pat. Nos. 4,683,195 and 4,683,202 such as, but not limited to, polymerase chain reaction (PCR), the reverse transcriptase PCR (RT-PCR) or the ligase PCR (LCR).

The standard nucleic acid should advantageously differ from the nucleic acid that is to be amplified in at least one detectable characteristic. In addition, it should advantageously be capable of amplification with the help of the same primer with which the target nucleic acid is amplified. Standard nucleic acids have proven to be practical when they have a different size (e.g., a different number of bases (b) or base pairs (bp) compared to the nucleic acid that is to be quantified or detected). In addition, the use of an additional restriction cleavage site has proven to be advantageous. Otherwise, it is preferable to make use, as the standard nucleic acid, of a nucleic acid that has the greatest possible similarity to the nucleic acid that is to be quantified or detected in the sample. This applies, in particular, to the GC content, the restriction sites, the sequence, etc. Preferred standards differ from the nucleic acid which is to be detected/quantified by at least 10% or 5 bases (base pairs) in terms of their length; in each case, however, these differences also depend on the quantification system for the amplified nucleic acids (for example, gel electrophoresis or a chromatographic process).

The detection and quantification of the amplified nucleic acids can then be undertaken in ways known to those of ordinary skill in the art such as a fluorescence sensitive nucleic acid detection apparatus (if use is made of fluorescent primers). Examples of such nucleic acid detection apparatus are automatic DNA sequencers with laser-induced fluorescence measurement devices, such as the Gene Scanner® 373A from the company Applied Biosystems, or HPLC devices. In the case of these types of apparatus, it is even possible to separate nucleic acid molecules from one another when these molecules differ by only one base pair in terms of their length. Such types of apparatus also permit the processing and analysis of a multitude of samples on a gel (e.g., by means of multiplex PCR).

The internal standard is preferably added to the sample prior to any possible pre-purification or extraction of the nucleic acid from the sample; as a result, false negative results that can arise from errors or losses from such pre-purification can be detected. Routine testing with use being made of 2 or more different internal standards (as described in Austrian patent specification number 401,062), which can also be used at different concentrations, can also be established simply and, in particular, inexpensively via the system in accordance with the invention.

Viral nucleic acids are, preferably, detected or quantified in accordance with the invention, especially those in samples that have been taken from body fluids or that serve as the starting product for medical preparations that are to be administered to humans. Typical clinical samples that are frequently tested using nucleic acid amplification assays include, but are not limited to blood, spinal fluid, semen, saliva and tears. Other samples may include, but are not limited to cell culture fluids, recombinant cells, and animal tissue and plant tissue.

The methodology in accordance with the invention has proven valuable especially in the case of viral nucleic acids that can otherwise be made available only via recombinant manipulation and cultivation of the standard. Preferred viruses which can be detected or quantified in accordance with the techniques of this invention are Transfusion-Transmitted Virus (TTV), human parvoviruses, especially parvovirus B19, hepatitis viruses, especially HAV, HBV and HCV, or retroviruses such as the human immunodeficiency virus (H IV).

Parvovirus B19 causes a disease in children, which usually proceeds in a mild fashion, namely infectious erythema. In immunocompromised persons, however, it can lead to erythema infectiosum and transient aplastic crisis in patients with hemolytic anemia, fetal death, arthritis and chronic anemia (Anderson: J. Infect. Dis. 161 (1990), pages 603-608). Transfusion-Transmitted Virus is a new DNA virus which was isolated recently in the serum of patients with post-transfusion hepatitis of unknown etiology (Simmonds et al.: Lancet 352 (1998), pages 191-194; Okamoto et al.: Hepatol. Res. 10 (1998), pages 1-16).

The present process is also particularly well suited to the characterization of nucleic acids or nucleic acid contamination from culture fluids, especially in a process such as that which is described in Austrian patent number 401,270 B.

The sample which is to be examined in accordance with the invention in this connection is advantageously derived from recombinant cells, tissue or animals, whereby the detection of contamination or genotyping represents prime usage areas in particular.

The invention will be elucidated in more detail by means of the following examples as well as by the figures, though it is not to be limited thereto.

### BRIEF DESCRIPTION OF THE DRAWINGS

FIG. 1: Single-stage internally controlled PCR (IC-PCR) for detection of Parvovirus B19. A sample containing Parvovirus B19 DNA was extracted, diluted in tenfold steps: 1 and 3.3 μL of the last two dilutions subjected to IC-PCR using primer pair KK5 SEQ ID NO: 1/KK6 SEQ ID NO: 2 (lanes 2-5). Additionally the same dilutions were mixed with approximately 10 copies internal control B19c SEQ ID NO: 8 and PCR amplified (lanes 6-9). 142 bp: fragment of wild type Parvovirus B19, 117 bp: fragment of internal control B19c SEQ ID NO: 8. Lane 1: negative control: lane 10: molecular weight marker (MspI digest of pBR322).

FIG. 2: IC-PCR for detection of TTV after co-extraction of samples and internal control. DNA was extracted from 200 μL plasma of 5 different pools of 32 healthy donors in the presence of approximately 50 copies internal control TTVc SEQ ID NO: 9. 15 μL of the extracted DNA solution was subjected to PCR using primer pair TTVS1 SEQ ID NO: 3/TTVA1 SEQ ID NO: 4. The PCR product of the internal control at 105 bp (lanes 1 and 3-5) indicated a successful PCR, a missing PCR product (lane 2) an inhibition of the PCR reaction. A PCR product of 286 bp shows a TTV positive sample (lane 1); lane 6: negative control, lane 7: molecular weight marker (MspI digest of pBR322).

FIG. 3: IC-PCR for genotyping of knockout mice. Tail snips were digested and 3 μL of the crude lysate were subjected to PCR alone or separately with both primer pairs in the presence of approximately 10 copies internal control FVIIIc SEQ ID NO:10. Primers MC18 SEQ ID NO: 5/MC19 SEQ ID NO: 6: lanes 1-6, primers MC18 SEQ ID NO: 5/neoR2 SEQ ID NO: 7: lanes 7-12. PCRs in lanes 2, 4, 8, 10 are performed without and in lanes 3, 5, 7, 9 in the presence of the internal control FVIIIc SEQ ID NO:10. A PCR product of 680 bp is from normal mice, 160 bp from knockout mice, the internal control FVIIIc SEQ ID NO: 10 yielded either 105 bp (MC18/MC19) or 85 bp (MC18/neoR2). Lanes 1-7: respective negative controls, lane 13: molecular weight marker (MspI digest of pBR322).

### DETAILED DESCRIPTION OF THE INVENTION

The terms “internal control,” “internal standard,” “standards nucleic acid” and “standard oligonucleotide” are used interchangeably. All of the aforementioned terms are meant to refer to the novel, chemically synthesized nucleic acid internal nucleic acid amplification assay control of the present invention.

### EXAMPLES

### Example 1

### IC-PCR For Detection and Quantitation of Parvovirus B19 and TTV and Genotyping of FVIII Knockout Mice

The internal controls (designated by a lower case “c” following the target identifier, for example, if the target is HIV, HIVc would designate the internal control nucleic acid) are custom synthesized oligonucleotides of a size of 105 nucleotides for TTVc SEQ ID NO: 9 and FVIIIc SEQ ID NO: 10 and 117 nucleotides for B19c SEQ ID NO:8 (MWG-BIOTECH GmbH, Ebersberg, Germany) containing the respective forward primer sequences and the complementary sequences of the respective reverse primer. The DNA sequence between the primer sequences was randomly chosen (sequences see Table 1).

### Extraction of DNA

Parvovirus B19 DNA was extracted from a sample containing B19 virus with the QIAGEN Blood kit (QIAGEN, Hilden, Germany) following the instructions of the supplier and the DNA was finally eluted with 50 μL H₂O. The indicated amounts of DNA were then subjected to PCR. For TTV, DNA was extracted from 200 μL citrated plasma of pools of 32 donors from an anonymous cohort of healthy subjects with the same procedure. In addition, prior the extraction, approximately 50 copies of the single-stranded internal control TTVc SEQ ID NO: 9 were added to the plasma pools. Finally 15 μL aliquots were subjected to PCR.

### Crude Lysates of Mouse Tail-snips

Pieces of tails approximately 5 mm in length were digested for 5 hours at 55° C. in 600 μL lysis buffer containing 10 mM Tris-HCl, pH 8.3, 2 mM MgCl₂, 0.01% Nonidet® P-40 and 200 μg/mL Proteinase K (Roche, Mannheim, Germany). The enzyme was inactivated for 10 min at 94° C., and 3 μL of this lysate was subjected directly to PCR.

### Polymerase Chain Reaction

One to three pL of tenfold dilutions of the B19 sample, 15 μL aliquots of the extracted TTV DNA samples or 3 μL of the tail-snip lysates were subjected to a single-stage IC-PCR employing a thermally activated DNA polymerase. IC-PCR was carried out in a total volume of 50 μL containing 1 Unit HotStarTaq™ (QIAGEN) in the respective buffer supplied by the manufacturer, 200 μM of each dNTP and 50 pmol each of primers of the respective primer pairs (SEQ ID NO: 1 and SEQ ID NO: 2 for B19 and SEQ ID NO 3 :SEQ ID NO: 4 for TTV. Internal controls were added to TTV samples already to the extraction procedure, to the B19 samples and to the tail lysates they were added to the indicated reaction tubes prior to PCR. The sequences of all primers and of the internal controls are shown in Table 1. Samples were then overlaid with mineral oil, incubated for 14 min at 94° C. and amplified for 45 cycles in a TRIO-Thermoblock (BioMetra, Gottingen, Germany) with the following cycle profile: 30 s at 94° C., 30 s at 55° C., 60 s at 72° C. with a final elongation of 72° C. for 1 min. The samples were fractionated on a 3.5% low-melting agarose gel stained with ethidium bromide.

### Results

It was previously reported that the use of a Taq DNA polymerase inactive at room temperature has a comparable sensitivity and specificity as nested PCR (Zimmermann et al. BioTechniques 24 (1998), page 222-224) which usually detects single copies of specific templates. In order to achieve highest sensitivity and specificity in a single-stage PCR protocol, a thermally activated DNA polymerase is used. To check the sensitivity of the single-stage PCR set-ups, calculated concentrations of the internal controls were endpoint diluted and repeatedly PCR amplified.

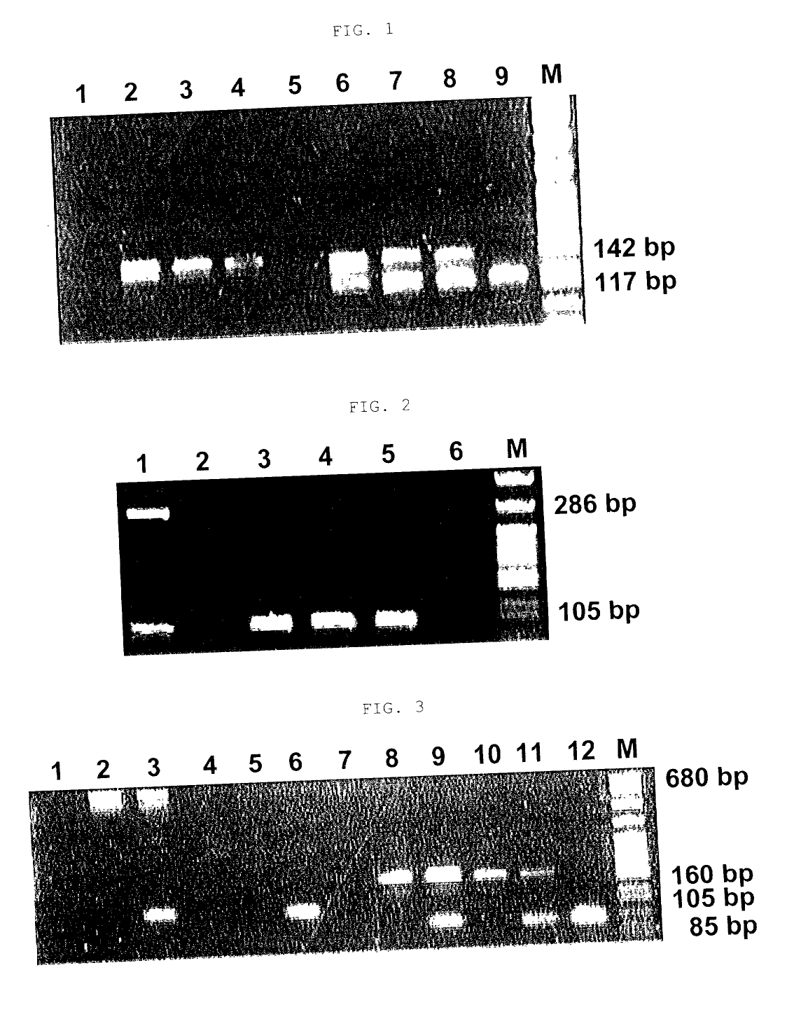

The principle of a single-stranded custom synthesized internal control was first checked in a PCR set-up for the detection of single-stranded DNA virus Parvovirus B19. DNA from a sample containing the virus was extracted and then diluted in tenfold steps to an endpoint. Then 1 and 3.3 μL of the last two dilutions were subjected to PCR. The same concentrations of the B19 sample were also mixed with approximately 10 copies internal control B19c SEQ ID NO: 8. This was the lowest amount necessary always leading to PCR amplification products. The samples were PCR amplified with primers KK5 SEQ ID NO: 1/KK6 SEQ ID NO: 2 located in the highly conserved region of B19 from bp 961 to bp 1102 (PVBAOA strain). FIG. 1 shows that the internal control produced a fragment of the expected 117 bp size (lanes 6-9). It should be noted that the lowest concentration of B19 visible without internal control (lane 4) was in this concentration influenced in it's intensity but still clearly visible if mixed with an amount of approximately 10 copies B19c SEQ ID NO: 8 (lane 8). To control extraction efficiency the internal control B19c SEQ ID NO:8 is added prior to a DNA extraction procedure.

The detection of TTV was chosen for demonstrating the usefulness of single-stranded oligonucleotides as internal control for a nucleic acid co-extraction procedure. Because TTV is frequently detected by PCR amplification using various PCR methods and primer sets which are all specific for the same region located in the ORF of TTV (Naoumov et al. Lancet 352 (1998), page 195-197; Nishizawa et al. BBRC 241 (1997), page 92-97; Okamoto et al.; Simmonds et al.). A primer set specific for this region (TTVSI SEQ ID NO:4/TTVA1 SEQ ID NO: 4) is chosen. Due to a recovery of less than 100% during DNA extraction approximately 50 copies of internal control TTVc SEQ ID NO:9 was the smallest possible number to be added prior to the TTV DNA extraction resulting in positive PCR amplification products. The internal control was added to 200 μL plasma consisting of pools of 32 donors, the samples were extracted and then subjected to IC-PCR. An example for typical IC-PCR experiments is shown in FIG. 2 where a TTV positive sample is shown in lane 1 and a missing PCR product of the internal control at lane 2 indicates an inhibition of the PCR. The PCR product of the internal control at 105 bp (lanes 1 and 3-5) indicates a successful PCR.

The examples of Parvovirus B19 and TTV demonstrate that single-stranded oligonucleotides are useful tools for IC-PCR, but it is still unclear if they could be used equally well in PCR set-ups analyzing double-stranded DNA. This PCR is also used for analysis of FVIII knockout mice as an example to check the tissue. The E-17 factor VIII-deficient mouse strain was produced by Bi et al. (Nat. Genet. 10 (1995), page 119-121; Blood 88 (1996), page 3446-3450) by insertion of a neomycin gene into the 3′ end of exon 17 of the factor Vil gene. One of the strategies for breeding is the crossbreeding of normal C57BL/6 females with semizygous affected knockout males (Muchitsch et al. Throm.Haem. 82(4) (1999), page 1371-1373).

To determine the zygosity of the offspring, crude lysates of tail snips are routinely genotyped. Especially to ascertain the heterozygosity of X′X females in crude lysates the addition of internal controls prior to PCR proved to be extremely helpful. An oligonucleotide containing the sequence of MC18 SEQ ID NO: 5 at the 5′ end and the complimentary sequences of ne2R SEQ ID NO: 7 and MC19 SEQ ID NO: 6 at the 3′ end (resulting after PCR in 85 and 105 bp fragments, respectively) was ordered. Thus, this internal control termed FVIIIc SEQ ID NO: 10 could be used both for amplification with either primer pair MC18/neoR2 resulting in a 160 bp fragment from factor Vil gene of knockout mice or for a separate amplification with MC18/MC19 of knockout mice or for a separate amplification with MC18/MC19 yielding a 680 bp fragment from factor VIII gene of normal mice.

Again, for crude lysates the addition of approximately 10 copies FVIIIc SEQ ID NO: 10 prior to PCR were the lowest number necessary always leading to PCR signals. FIG. 3 shows a typical example of a genotyping experiment performed with two chosen samples of X′X females. Both samples were amplified alone or in the presence of the internal controls with either primer pair MC18 SEQ ID NO: 5/MC19 SEQ ID NO: 6 (lanes 1-6) or MC18 SEQ ID NO:5/neoR2 SEQ ID NO: 7 (lanes 7-12). Sample 1 (lanes 2, 3, 8, 9) showed all expected bands of the specific template and the internal control with both primer pairs whereas sample 2 (lanes 4, 5,10,11) containing obviously PCR inhibitors resulted in only faint bands with MC18/neoR2 (lanes 10 and 11) and in no bands with MC18/MC19 (lanes 4 and 5). Only the use of an internal control avoided a false determination of the genotype.

### Example 2

### Serial Testing of TTV According to the Present Invention with a Comparative Test to Nexted PCR and Semi-nested PCR

### Methods

### Extraction of DNA

Citrated plasma samples were collected from an anonymous cohort of healthy plasma donors. All were HBV, HCV and HIV-1 negative regular donors. DNA was extracted from 200 μL plasma of a pool of 32 donors with the QIAGEN Blood kit (QIAGEN, Hilden, Germany) following the instructions of the supplier. In addition, prior to the extraction procedure, approximately 50 copies of the single-stranded internal control iTVc SEQ ID NO:9 were added to the plasma pools and the DNA was finally eluted with 50 μL H₂O. The internal control is custom synthesized 105 base oligonucleotide (MWG-BIOTECH GmbH, Ebersberg, Germany) containing the primer sequence of TTVS1 SEQ ID NO: 3and the complementary sequence of TTVA1 SEQ ID NO: 4.

### Polymerase Chain Reaction

The extracted DNA solution of the plasma pools was divided in 15 μL aliquots and subjected either to nested PCR as described by Simmonds et al., to semi-nested PCR as described by Okamoto et al., (Taq DNA polymerase and 10× buffer from Pharmacia, Uppsala, Sweden), or to single-stage IC-PCR employing a thermally activated DNA polymerase. IC-PCR was carried out in a total volume of 50 μL containing 1 Unit of HotStarTaqTM (QIAGEN) in the respective buffer supplied by the manufacturer, 200 μM of each dNTP and 50 pmol each of forward primer TTVS1 SEQ ID NO: 3 and reverse primer TTVA1 SEQ ID NO:4. The sequence of all primers and of the internal controls are shown in Table 1. Samples were overlaid with mineral oil, incubated for 14 min at 94° C. and amplified for 45 cycles in a TRIO-Thermoblock (BioMetra, Gottingen, Germany) with the following cycle profile: 30 s at 94° C., 30 s at 55° C., 60 s at 72° C. with a final elongation at 72° C. for 1 min. The samples were then fractionated on a 3.5% low-melting agarose gel stained with ethidium bromide. Amplification of a positive sample with primer pairs TTVS1 SEQ ID NO:3-TTVA1 SEQ ID NO: 4 resulted in the expected 286 bp PCR product and of the internal control in a 105 bp band.

### Results

In order to achieve the highest sensitivity and specificity in a single-stage PCR protocol, HotStarTaq™ was used. To determine the sensitivity of this single-stage PCR set-up, the internal control, a simple, custom synthesized 105 base oligonucleotide, was endpoint diluted and repeatedly PCR amplified. Taking into account the Poisson distribution, the sensitivity of our assay was confirmed to be on the single copy level. The IC-PCR is compared with the nested and semi-nested PCR protocols most frequently used at the time for amplification of the same TTV region (Charlton et al. Hepatology 28 (1998), page 839-842; Hohne et al. J. Gen. Virol. 79 (1998), page 2761-2764; Naoumov et al., Nishizawa et al., Okamoto et al., Prescott et al. NEJM 339 (1998), page 776-777; Simmonds et al., Tanaka et al. J. Med. Virol. 56 (1998), page 234-238 and FEBS Letters 437 (1998), 201-206). Considering the previous data (Simmonds et al.), a low prevalence in a European population for this region of TTV was expected and therefore compared 20 different pools consisting of the plasma of 32 donors to obtain more positive samples.

Approximately 50 copies of the internal control (the lowest number necessary always leading to PCR signals) were added to 200 pL plasma, the sample was extracted and then subjected to IC-PCR, to the nested PCR as described by Simmonds et al. and to the semi-nested PCR as described by Okamoto et al. All samples were independently extracted and subjected to the different PCRs (including positive and negative control) three times. The results are summarized in Table 3. The comparison of the three methods shows a comparable sensitivity and specificity of our method and the method of Simmonds (13/20 vs 11/20 samples positive), whereas in our hands, the PCR set-up as described by Okamoto et al. did not work. From 60 IC-PCRs two reactions were inhibited once (samples 1 and 18) and the two other tests were positive. In contrast to IC-PCR, in these two cases, the conventional PCR would have given false-negative results. Samples 3, 4 and 12 always showed the band of the internal control, but were only positive in two of three PCR experiments, which is probably due to the low copy number of TTV often present in plasma (PCR methods for TTV are obviously at the border of delectability).

TABLE 1

Sequences of Primers and Internal Controls

| | | | | Length of |
|---|---|---|---|---|
| Target | Sequence | Oligotype | Primer Sequence | fragment |
| Parvo B19 | KK5 | Forward | SEQ ID NO:1 | 142 bp |
| | | | 5′-CCAAGAAACCCCGCATTACC-3′ | |
| | KK6 | Reverse | SEQ ID NO:2 | |
| | | | 5′-ACCAGUTACCATAGTTTGAA-3′ | |
| TTV | TTVS1 | Forward | SEQ ID NO 3 | 286 bp |
| | | | 5′-ACAGACAGAGGAGAAGGCAAC-3′ | |
| | TTVA1 | Reverse | SEQ ID NO:4 | |
| | | | 5′-CTGGCATTTTACCATTTCCAA-3′ | |
| FVIII | MC-18 | Forward | SEQ ID NO:5 | approx. |
| knockout | | | 5′-GAGCAAATTCCTGTACTGAC-3′ | 680 bp |
| | MC-19 | Reverse | SEQ ID NO:6 | (MC18/MC |
| | | | 5′-TGCAAGGCCTGGGCTVATTT-3′ | /19) |
| | neoR2 | Reverse | SEQ ID NO:7 | approx. |
| | | | 5′-CCGCCCTCCCTTGCGCTAC-3′ | 160 bp |
| | | | | (MC18/neo |
| | | | | R2) |
| Parvo 19 | B19c | Internal | SEQ ID NO 8: | 117 bp |
| | | Control | 5′-GCCAAGAAACCCCGCATTAC | |
| | | | CATGTTATGGATAGACTGGC | |
| | | | TAAGCAAAGCGCGATCCAAA | |
| | | | ACACAAAAGGCTTTGTTCCT | |
| | | | TACTCTTTAAACTTTGTTCA | |
| | | | AACTATGGTAAACTGGT-3′ | |
| TTV | TTVc | Internal | SEQ ID NO:9 | 105 bp |
| | | Control | 5′-ACAGACAGAGGAGAAGGCAA | |
| | | | CATGTTATGGATAGACTGGC | |
| | | | TAAGCAAAAAAACACAAAAG | |
| | | | GCTTTGTVCCTTACTCTTTA | |
| | | | AACTTTGGAAATGGTAAAAT | |
| | | | GCCAG-3′ | |
| FVIII | FVIIIc | Internal | SEQ ID NO:10 | 105 bp |
| knockout | | Control | 5′-GAGCAAATVCCTGTACTGAC | (MC18/MC |
| | | | CATGTTATGGATAGACTGGC | 19) |
| | | | TAAGCAAAGCGCGATCCAAA | 85 bp |
| | | | ACACAAGTAGCGCAAGGGAG | (MC18/neo |
| | | | GGCGGAAATAAGCCCAGGCC | R2) |
| | | | TTGCA-3′ | |

TABLE 2
Sequences of Primers and Internal Control

| | | |
|---|---|---|
| NG059 | SEQ ID NO:11 | 5′-ACAGACAGAGGAGAAGGCAACATG-3′ |
| NG061 | SEQ ID NO:12 | 5′-GGCAACATGTTATGGATAGACTGG-3′ |
| NG063 | SEQ ID NO:13 | 5′-CTGGCATTTTACCATTTCCAAAGTT3′ |
| A5430 | SEQ ID NO:14 | 5′-CAGACAGAGGAGAAGGCAACATG-3′ |
| A5427 | SEQ ID NO:15 | 5′-TACCAYTTAGCTCTCATTCTWA-3′ |
| A8761 | SEQ ID NO:16 | 5′-GGMAAYATGYTRTGGATAGACTGG-3′ |

TABLE 3
Comparison of Three Different PCR Methods for the Detection of TTV

| Sample | IC-PCR | | | Nested | | | Semi-nested | | |
|---|---|---|---|---|---|---|---|---|---|
| No. | 1 | 2 | 3 | 1 | 2 | 3 | 1 | 2 | 3 |
| 1 | + | + | i | + | + | − | − | − | − |
| 2 | + | + | + | + | + | + | − | − | + |
| 3 | + | − | + | − | − | − | − | − | − |
| 4 | − | + | + | − | − | − | − | − | − |
| 5 | + | + | + | + | + | + | − | − | − |
| 6 | + | + | + | + | + | − | − | − | − |
| 7 | + | + | + | + | + | + | + | + | + |
| 8 | − | − | − | − | − | − | − | − | − |
| 9 | − | − | − | − | − | − | − | − | − |
| 10 | + | + | + | + | + | + | − | + | − |
| 11 | − | I | − | − | − | − | − | − | − |
| 12 | − | + | + | − | + | − | − | − | − |
| 13 | + | + | + | + | + | + | − | − | − |
| 14 | − | − | − | − | − | − | − | − | − |
| 15 | − | − | − | − | − | − | − | − | − |
| 16 | + | + | + | + | + | + | − | − | − |
| 17 | − | − | − | − | − | − | − | − | − |
| 18 | i | + | + | − | + | + | − | − | − |
| 19 | + | + | + | + | + | + | − | − | − |
| 20 | − | − | − | − | − | − | − | − | − |

Each sample (pools of 32 donors) was extracted three times and each extracted solution subjected to the three different PCR set-ups: + = positive, − = negative, i = inhibition of reaction

Reference has been made herein to various patents, printed publications and manufacture instructions for use. Each of the aforementioned references is incorporated herein by reference in their entirety.

What is claimed is:

1. An internal control for a nucleic acid amplification assay comprising:
a synthetic nucleic acid wherein said internal control is made using non-recombinant DNA techniques.

2. The internal control of claim 1 wherein said non-recombinant DNA technique is chemical synthesis.

3. The internal control of claim 1 herein said non-recombinant DNA technique uses a oligonucleotide synthesizer.

4. The internal control of claim 1 wherein said synthetic nucleic acid is single stranded DNA.

5. The internal control of claim 1 wherein said synthetic nucleic acid is single stranded RNA.

6. A method for the detection of nucleic acids in a sample, comprising:
providing a sample;
adding at least one internal nucleic acid standard to the sample;
amplifying nucleic acids present in said sample; and
detecting said amplified nucleic acids;
wherein said at least one internal standard is an oligonucleotide synthesized using non-recombinant DNA techniques.

7. The method for the detection of nucleic acids in a sample of claim 6 wherein said method is a quantitative polymerase chain reaction (PCR) assay.

8. The method for the detection of nucleic acids in a sample of claim 6 wherein said internal nucleic acid standard differs from a target nucleic acid to be detected and wherein said internal standard and said target nucleic acid have at least one detectable difference.

9. The method for the detection of nucleic acids in a sample of claim 6 wherein said nucleic acids are extracted prior to amplifying said nucleic acids in said sample, and whereby said internal nucleic acid standard is added prior to said extraction of said nucleic acids in said sample.

10. The method for the detection of nucleic acids in a sample of claim 8 wherein said target nucleic acid is viral nucleic acid.

11. The method for the detection of nucleic acids in a sample of claim 10 wherein said viral nucleic acid is selected from the group consisting of transfusion transmissible virus nucleic acid, parvoviruses nucleic acid, hepatitis B virus nucleic acid, hepatitis C virus nucleic acid, and human immunodeficiency virus nucleic acid.

12. The method for the detection of nucleic acids in a sample of claim 6 wherein said sample is selected from the group consisting of blood, spinal fluid, semen, saliva and tears.

13. The method for the detection of nucleic acids in a sample of claim 6 wherein said sample is cell culture fluid.

14. The method for the detection of nucleic acids in a sample of claim 6 wherein said sample is selected from the group consisting of recombinant cells, animal tissue and plant tissue.

15. The method for the detection of nucleic acids in a sample of claim 8 wherein said internal nucleic acid standard differs from said target nucleic acid in terms of its length.

16. The method for the detection of nucleic acids in a sample of claim 15 wherein said internal nucleic acid standard differs from said nucleic acid by at least 10% or 5 bases (base pairs) in terms of its length.
